(19) United States
(12) Patent Application Publication
(10) Pub. No.: US 2024/0324856 A1
(43) Pub. Date: Oct. 3, 2024

(54) SURGICAL TROCAR WITH INTEGRATED CAMERAS

(71) Applicant: Covidien LP, Mansfield, MA (US)

(72) Inventor: William J. Peine, Ashland, MA (US)

(21) Appl. No.: 18/576,151

(22) PCT Filed: Jul. 26, 2022
(86) PCT No.: PCT/IB2022/056859

§ 371 Date: Jan. 3, 2024

Related U.S. Application Data
(60) Provisional application No. 63/228,185, filed on Aug. 2, 2021.

(51) Int. Cl. A61B 1/00; A61B 1/05; A61B 1/06; A61B 17/34; A61B 34/30

(52) U.S. Cl. CPC A61B 1/00149; A61B 1/00009; A61B 1/0005; A61B 1/05; A61B 1/0676; A61B 1/0684; A61B 17/3421; A61B 34/30; A61B 2034/302

(57) ABSTRACT

A surgical robotic system for use in a minimally invasive surgical procedure includes a trocar for facilitating passage of a surgical instrument into a body cavity and capturing images of the body cavity.

### FIELD

The present technology is generally related to surgical robotic systems used in minimally invasive medical procedures.

### BACKGROUND

Some surgical robotic systems include a console supporting a surgical robotic arm and a surgical instrument or at least one end effector (e.g., forceps or a grasping tool) mounted to the robotic arm. The robotic arm provides mechanical power to the surgical instrument for its operation and movement. Each robotic arm may include an instrument drive unit operatively connected to the surgical instrument and coupled to the robotic arm via a rail. In operation, the robotic arm is moved to a position over a patient and then guides the surgical instrument into a small incision via a surgical trocar or a natural orifice of a patient to position the end effector at a work site within the patient's body. The surgical trocar may be attached to an end of the surgical robotic arm and held in a fixed position during insertion of the surgical instrument therethrough.

It would be advantageous to provide better visualization within the patient's body during surgical instrument insertion and usage of the surgical instrument.

### SUMMARY

In one aspect of the disclosure, a surgical robotic system is provided and includes a surgical robotic arm and a first trocar. The surgical robotic arm has an elongated rail configured to movably support a surgical instrument. The first trocar includes a head configured for attachment to the elongated rail, a cannula extending distally from the head and configured to receive the surgical instrument, and a plurality of cameras disposed about a distal end portion of the cannula and directed radially outward.

In aspects, the surgical robotic system may further include a video processing device and a display in communication with the video processing device. The video processing may be in communication with the cameras of the first trocar and may be configured to stitch together images taken by each of the cameras of the first trocar to form a single image. The display may be configured to display the single image.

In aspects, the cameras may be mounted to the distal end portion of the cannula in an annular array.

In aspects, the first trocar may include a lens enclosing the plurality of cameras.

In aspects, the distal end portion of the cannula may define a distal port, and the cameras may be disposed adjacent the distal port.

In aspects, the first trocar may include a light disposed adjacent the cameras.

In aspects, the surgical robotic system may further include a second trocar that includes a cannula defining a channel therethrough, and a plurality of cameras disposed about a distal end portion of the cannula of the second trocar and directed radially outward.

In aspects, the video processing device may be further configured to focus the cameras of the first trocar or the second trocar on the other of the first trocar or the second trocar during insertion of the surgical instrument into the other of the first trocar or the second trocar.

In aspects, the surgical robotic system may further include a display and a video processing device in communication with the display and the cameras of the first trocar. The video processing device may be configured to stitch together images taken by each of the cameras of the first trocar to form a single image and display the single image on the display.

In accordance with another aspect of the disclosure, a trocar for insertion into a body cavity is provided and includes a head defining an opening configured for receipt of a surgical instrument, a cannula extending distally from the head and defining a channel configured for passage of the surgical instrument, and a plurality of cameras disposed about a distal end portion of the cannula and directed radially outward.

In aspects, the distal end portion of the cannula may define a distal port, and the cameras may be mounted to the distal end portion of the cannula adjacent the distal port.

In aspects, the cannula may have a proximal end portion attached to the head, and the distal end portion of the cannula may have a distal tip configured for penetrating tissue.

In aspects, the cannula may include a lens enclosing the plurality of cameras.

In accordance with another aspect of the disclosure, a method of imaging an internal body cavity during a surgical procedure is provided. The method includes stitching images captured by a plurality of cameras disposed about a distal end portion of a first trocar to form a single image of a body cavity; and displaying the single image of the body cavity on a display.

In aspects, the method may further include activating the cameras of the first trocar as a second trocar is inserted into the body cavity and/or directing the cameras of the first trocar at the second trocar as the second trocar is inserted into the body cavity.

In aspects, the method may further include stitching together images taken by the cameras of the first trocar and a plurality of cameras of the second trocar to form a 3D image of the body cavity; and display the 3D image on the display.

In aspects, the method may further include detecting movement of a surgical instrument into the body cavity, whereupon the plurality of cameras of the first trocar and a plurality of cameras of the second trocar are oriented toward the surgical instrument.

In aspects, the method may further include illuminating the body cavity with a plurality of LEDs mounted to the distal end portion of the first trocar.

Further details and aspects of exemplary aspects of the disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the disclosure are described herein with reference to the accompanying drawings, wherein:

FIG. 1 is a schematic illustration of a surgical robotic system including a control tower, a console, and one or more surgical robotic arms;

FIG. 2 is a perspective view of a surgical robotic arm of the surgical robotic system of FIG. 1;

FIG. 3 is a perspective view of a setup arm with the surgical robotic arm of the surgical robotic system of FIG. 1;

FIG. 4 is a schematic diagram of a computer architecture of the surgical robotic system of FIG. 1;

FIG. 5 is a perspective view illustrating a surgical trocar of the surgical robotic system of FIG. 3;

FIG. 6 is a front view of a distal end portion of the trocar of FIG. 5 shown enlarged to illustrate a plurality of cameras of the trocar;

FIG. 7 is a front view of the trocar illustrating a vertical viewing angle of one of the cameras thereof;

FIG. 8 is a bottom view of the trocar illustrating a horizontal viewing angle of one of the cameras thereof;

FIG. 9 is a bottom view of the trocar illustrating the horizontal viewing angles of each of the cameras thereof;

FIG. 10 is a perspective view of the trocar illustrating a composite image of each of the images captured by the cameras thereof;

FIG. 11 is a perspective view of the trocar of FIG. 5 illustrating the trocar being inserted into a body cavity;

FIG. 12 is a perspective view illustrating three different angular positions of the trocar of FIG. 5 relative to the body cavity;

FIGS. 13A-13C illustrate three perspective views of a plurality of trocars within the body cavity and the viewing angles of each;

FIG. 14 is a perspective view illustrating the plurality of trocars of FIG. 13A-13C positioned within the body cavity simultaneously; and

FIG. 15 is a flow chart illustrating an exemplary method of utilizing the surgical robotic system of FIG. 1.

### DETAILED DESCRIPTION

Embodiments of the disclosed surgical robotic system are described in detail with reference to the drawings, in which like reference numerals designate identical or corresponding elements in each of the several views. As used herein the term “distal” refers to that portion of the surgical robotic system or component thereof, that is closer to a patient, while the term “proximal” refers to that portion of the surgical robotic system or component thereof, that is further from the patient.

One of the challenges of performing laparoscopic surgery is maintaining awareness of what is happening in the entire abdominal or thoracic cavity. This is often a result of the fact that laparoscopes have a limited viewing angle and surgeons zoom in on the immediate surgical site.

This disclosure describes a surgical trocar or “port” that contains a plurality of cameras attached at the distal end to provide more complete visualization of the internal cavity of the patient and to improve safety as additional ports or instruments are inserted during the procedure.

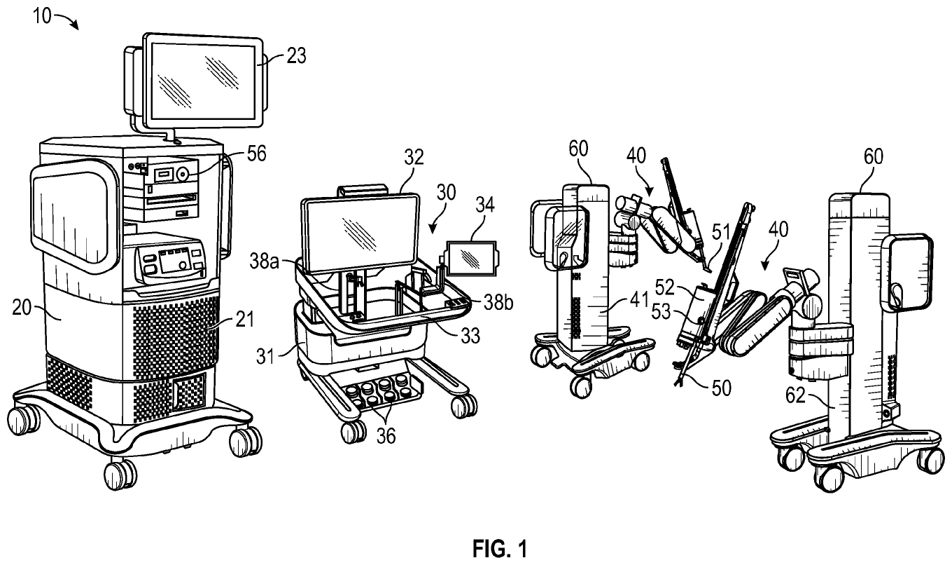

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

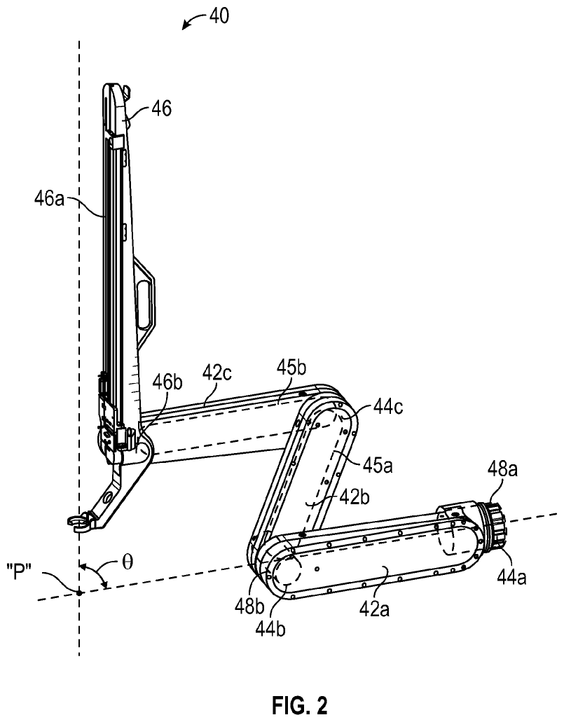

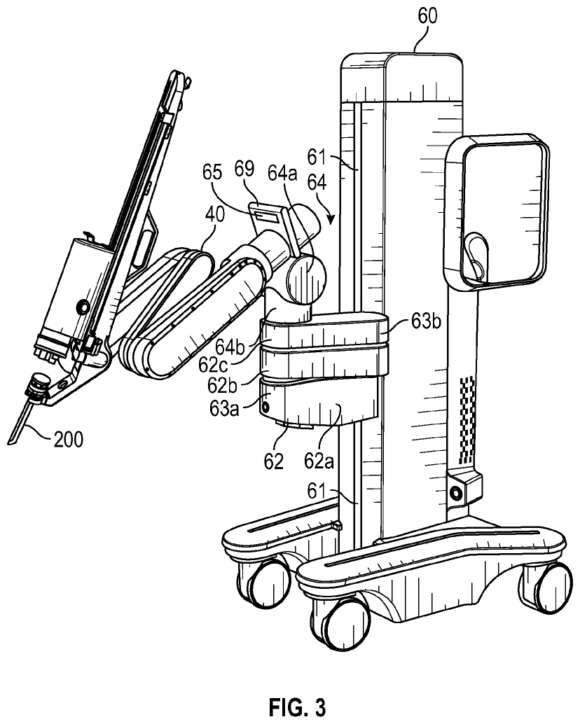

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port or surgical trocar 200 (FIG. 3) held by the holder 46.

The robotic arm 40 also includes a plurality of manual override buttons 53 disposed on the IDU 52 and the setup arm 62, which may be used in a manual mode. The user may press one or more of the buttons 53 to move the component associated with the button 53.

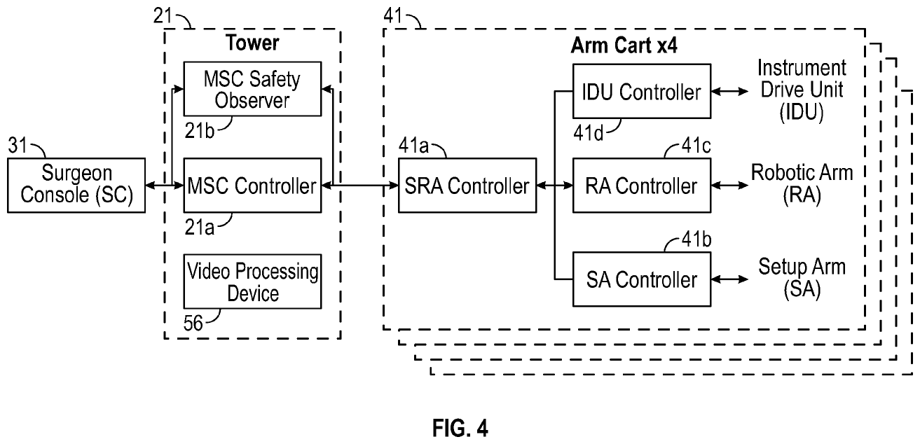

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

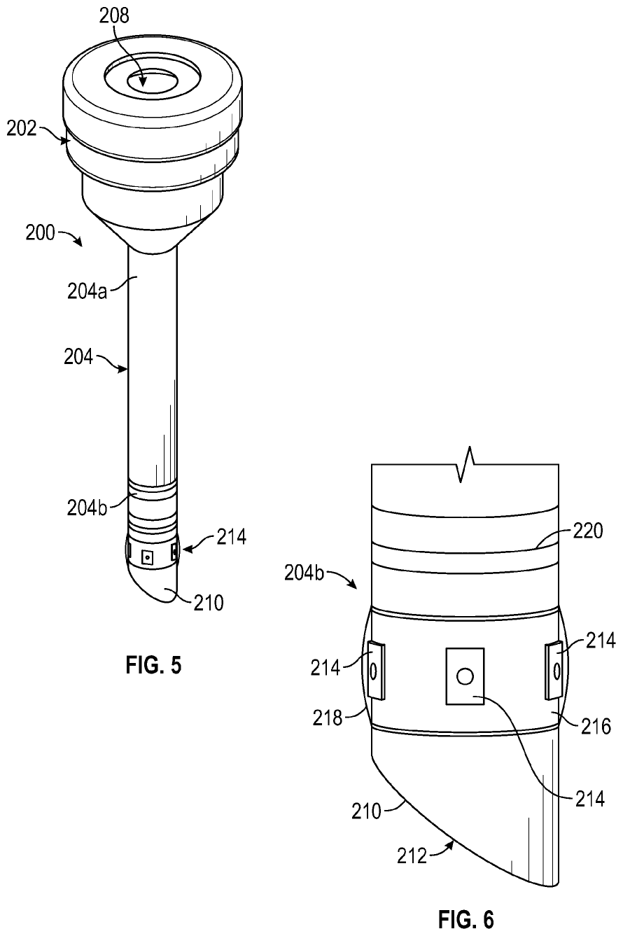

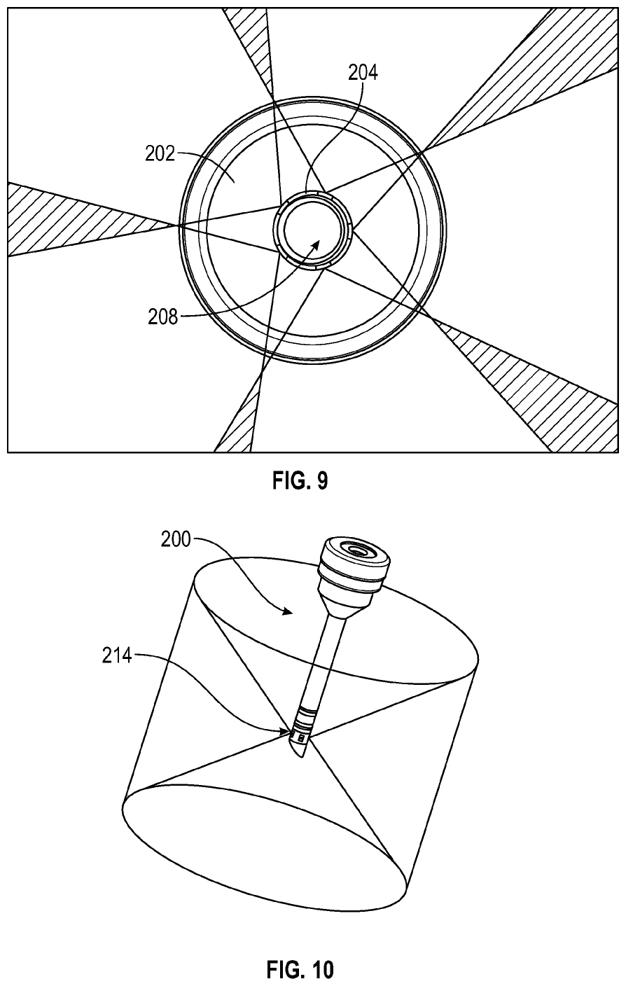

With reference to FIGS. 5-15, the surgical trocar 200 of the surgical robotic system 10 is configured for guiding the surgical instrument 50 through a natural or artificial opening in a patient and into a surgical site or internal body cavity of the patient. The trocar 200 generally includes a head 202 and a cannula 204 extending distally from the head 202. The head 202 and cannula 204 collectively define a channel 208 longitudinally therethrough configured to receive the surgical instrument 50. The cannula 204 has a proximal end portion 204a monolithically formed with or otherwise attached to the head 202, and a distal end portion 204b having a distal tip 210 defining a distal port 212 therein. The distal tip 210 may be set at an oblique angle relative to a longitudinal axis of the cannula 204 and may be sharp for traumatic insertion of the trocar 200 into tissue.

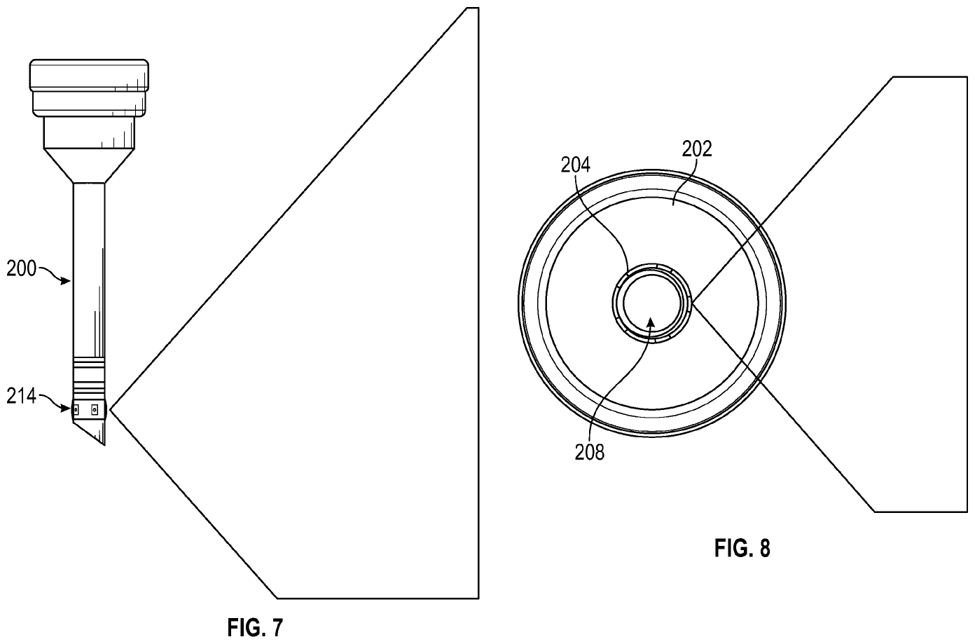

The trocar 200 further includes a plurality of cameras 214 disposed about the distal end portion 204b of the cannula 204 adjacent the distal port 212. In aspects, only a single camera 214 may be disposed at the distal end portion 204b of the cannula 204. The cameras 214 may be directed radially outward from an outer surface 216 of the cannula 204 and circumferentially spaced from one another about the distal end portion 204b of the cannula 204. The cameras 214 may be mounted to the outer surface 216 of the cannula 204, embedded in the outer surface 216 of the cannula 204, movably coupled to the cannula 204, or otherwise coupled to the distal end portion 204b of the cannula 204. In aspects, the cameras 214 may be solid-state imaging devices such as a Charge Coupled Device (CCD) type imaging device or a Complementary Metal Oxide Semiconductor (CMOS) type imaging device or any other suitable type of imaging device. Each of the cameras 214 may have a lens assembly (not explicitly shown) that provides a vertical viewing angle (FIG. 7) and a horizontal viewing angle (FIG. 8) of at least 90 degrees and up to about 180 degrees such that collectively the cameras provide a 360 degree view around the trocar 200, as shown in FIGS. 9-11. In aspects, the entire assembly of cameras 214 may be encapsulated or covered by a single lens 218 (FIG. 6), such as, for example, a fisheye lens that wraps around the distal end portion 204b of the cannula 204. In aspects, the cameras 214 may be configured to move between a retracted position and an extended position, in which the cameras 214 protrude outwardly for downward viewing of a surgical instrument being inserted through the trocar 200. The cameras 214 may be attached to the cannula 204 via a biasing mechanism that when actuated moves (e.g., springs out) the cameras 214 from the retracted position to the extended position.

The cameras 214 are in wired or wireless communication with the video processing device 56 of the tower 20. In other aspects, the cameras 214 may be in wired or wireless communication with at least one of the processors of the computers 21, 31, or 41 (FIG. 4) of the surgical robotic system 10. Wireless communication includes radio frequency, optical, WIFI, Bluetooth® (an open wireless protocol for exchanging data over short distances (using short length radio waves) from fixed and mobile devices, ZigBee® (a specification for a suite of high level communication protocols using small, low-power digital radios based on the IEEE 802.15.4-2003 standard for wireless personal area networks (WPANs)), etc.

The trocar 200 may further include a plurality of illumination devices or light sources, i.e., LEDs 220 (FIG. 6) disposed adjacent the cameras 214 or incorporated into the cameras 214. The light sources 220 may be disposed in an annular array about the distal end portion 204b of the cannula 204. The light sources 220 may be light-emitting diodes (LEDs) for illuminating the fields of view. In aspects, white light LEDs or other colors of LEDs or any combination of LEDs may be used, such as, for example, red, green, blue, infrared, near infrared and ultraviolet or any other suitable LED.

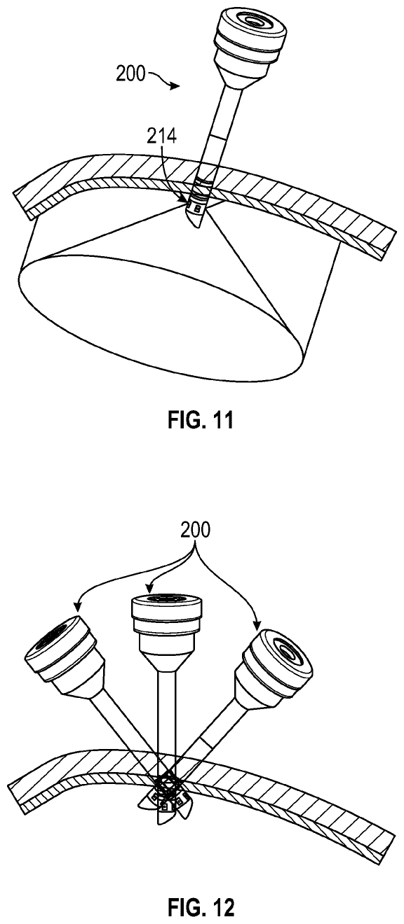

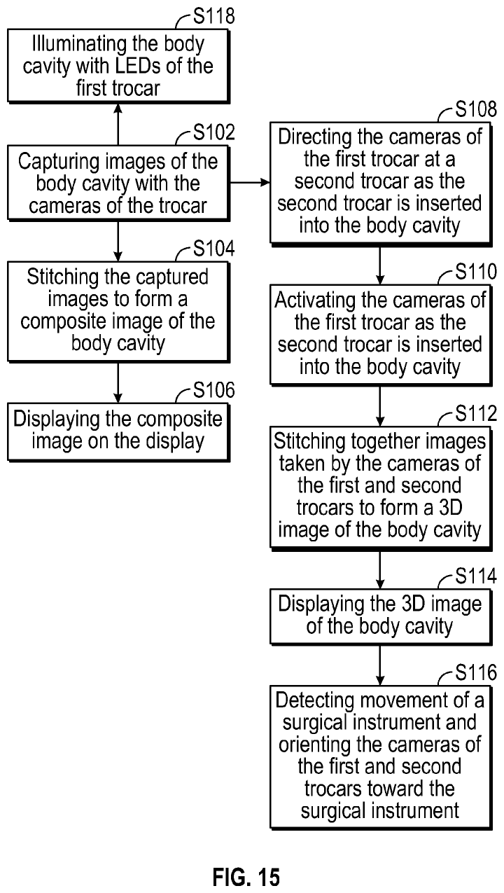

FIG. 15 illustrates a flow chart depicting an exemplary method of imaging a body cavity of a patient (e.g., a thoracic cavity or an abdominal cavity) utilizing the surgical robotic system 10 described herein. The first trocar 200 is inserted through tissue and into a body cavity of a patient. With at least the distal end portion 204b of the cannula 204 of the first trocar 200 positioned within the body cavity, in step S102 of the method, the cameras 214 of the first trocar 200 are activated, whereby the cameras 214 capture images of the body cavity and transmit the captured images to the video processing device 56. As shown in FIGS. 9-11, each of the viewing angles of the cameras 214 overlap to provide the 360 degree view around the trocar 200. In step S104, the video processing device 56 executes instructions stored in the memory of one of the computers 21, 31, 41 to stich the images captured by each of the cameras 214 to form a single composite image (e.g., a 360 degree panoramic image) of the body cavity, as shown in FIG. 11. In step S106, the video processing device 56 may transmit the single image to one or more of the displays 23, 32, or 34 (FIG. 1) for viewing by a clinician. As shown in FIG. 12, the trocar 200 may be manipulated (e.g., pivoted about a remote center of motion) either manually or via the surgical robotic arm 2 to adjust the viewing angles of the cameras 214.

The LEDs 220 of the trocar 200 may be activated to illuminate the body cavity while the cameras 214 are capturing the images and/or during insertion of the trocar 200. In aspects, the LEDs 220 may be a specific frequency to enable fluorescence imaging or contrast-based (e.g., indocyanine green dye) imaging. The LEDs 220 may be configured to change colors or flash for better identification. The flashing or changing of color of the LEDs 220 may be synchronized with a GUI on one or more of the displays 23, 32, or 34.

With the trocar 200 positioned within the body cavity, the surgical instrument 50 may be guided through the trocar 200 and into the body cavity to perform a surgical procedure.

In aspects, software may be provided in one of the computers 21, 31, or 41 to zoom in on a region of interest within the 360 degree view of the cameras 214. The software may also correct for distortion and allow the surgeon to pan around the body cavity. During instrument exchange, the software may also automatically zoom in on the trocar 200, 300, or 400 that has the surgical instrument 50 moving therethrough. This may help ensure that surgical instrument 50 does not puncture tissue along the path of the trocar 200, 300, or 400.

In aspects, one or more of the displays 23, 32, or 34 may have a user interface that allows the clinician to pan and zoom in to any region of the displayed image. This may allow viewing angles to be named or stored for quick switching between viewpoints, or recorded during the procedure. In aspects, image processing algorithms may be provided to track and follow a tip of the surgical instrument 50 as it is extracted or inserted. The image processing algorithms may also include real-time corrections, such as color enhancement or smoke removal.

In aspects, “follow me mode” type algorithms may be provided to move the zoomed in region-of-interest to automatically track an instrument or organ. The follow me mode may automatically compensate for the motion of the trocar to keep the region of interest centered on the frame.

In aspects, the trocar 200 may also include an inertial measurement unit (IMU) or gyro to provide automatic compensation for trocar motion during standard laparoscopic surgery when there is no robot to inform the clinician how the trocar is moving.

In aspects, when multiple of the disclosed trocars are used at the same time, an algorithm may be provided that selects the view from one of the trocars that is not actively being moved during teleoperation.

In other aspects, a tubular insert may be provided that is configured to be passed through a standard trocar or one of the trocars disclosed herein. The tubular insert may have cameras disposed about a distal end portion thereof and an integrated valve at a proximal end portion thereof. A surgical instrument may be inserted through the tubular insert.

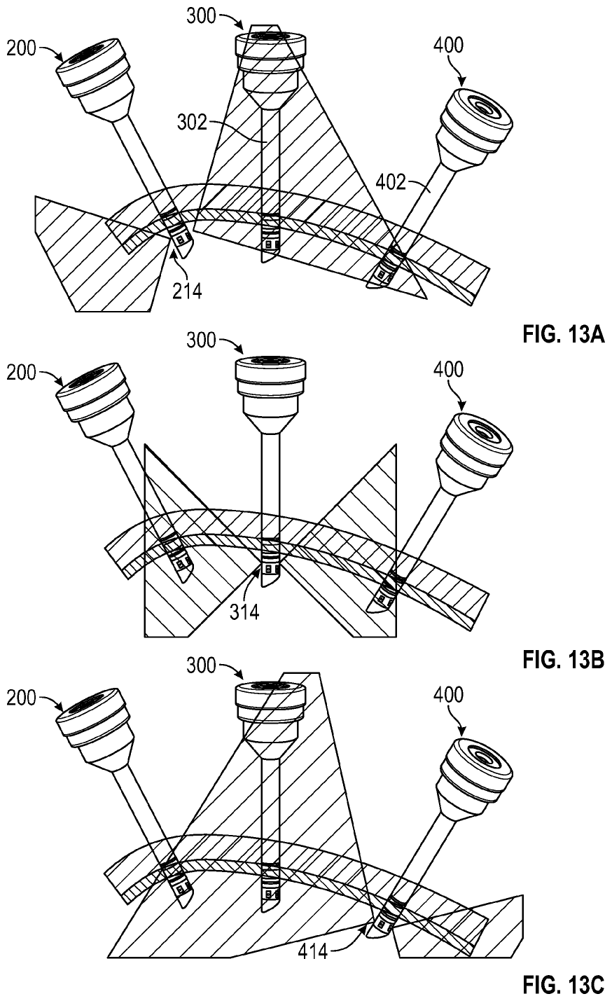

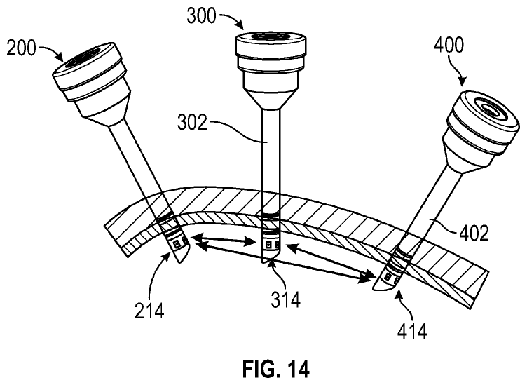

With reference to FIGS. 13 and 14, the surgical robotic system 10 may further include a plurality of secondary trocars 300, 400, each similar to the first trocar 200. More specifically, each of the secondary trocars 300, 400 respectively include a cannula 302, 402 and a plurality of cameras 314, 414 disposed about a distal end portion thereof and directed radially outward. The video processing device 56 and/or the processor of either of the computers 21, 31, or 41 (FIG. 4) of the surgical robotic system 10 is in communication with the cameras 314, 414 of the secondary trocars 300, 400. The video processing device 56 may be configured to stitch together images taken by each of the plurality of cameras 214, 314, 414 of each of the trocars 200, 300, 400 to form a single image and display the single image on one or more of the displays 23, 32, or 34 (FIG. 1). For details about the process of stitching images, reference may be made to U.S. Pat. No. 8,416,282, the entire contents of which are hereby incorporated by reference herein.

As shown in FIGS. 13A-13C, the secondary trocars 300, 400 may also be inserted through the tissue and into the body cavity. During the discrete insertion of each of the secondary trocars 300, 400, in steps S108, S110, respectively (FIG. 15), the cameras 214 of the first trocar 200 are directed or oriented toward the secondary trocar 300 or 400 and activated as the secondary trocar 300 or 400 is being inserted to assist a clinician in avoiding critical structures within the body cavity during insertion of each of the trocars 300, 400. As shown in FIG. 14, the cameras 214, 314, 414 of each trocar 200, 300, 400 may be used to view and localize the other trocars 200, 300, 400 to assist with instrument exchange and the localization of the trocars 200, 300, 400 relative to one another.

In aspects, the cameras 314, 414 of each of the secondary trocars 300, 400 and the cameras 214 of the first trocar 200 may capture images of the body cavity. In step S112 (FIG. 15, the video processing device 56 may stitch together the images taken by the cameras 214 of the first trocar 200 and the cameras 314, 414 of the secondary trocars 300, 400 to form a 3D image of the body cavity. In step S114, the 3D image of the body cavity may be displayed on one or more of the displays 23, 32, or 34 for viewing by the clinician. In step 116, the motion sensors of the trocars 200, 300, 400 detect movement of a surgical instrument (e.g., a surgical stapler, a vessel sealer, another trocar, etc.) into the body cavity, whereupon the plurality of cameras 214, 314, 414 of the trocars 200, 300, 400 are oriented toward the surgical instrument. In step S118, the body cavity may be illuminated with the LEDs 220 of the first trocar 200.

In aspects, when trocars 200, 300, 400 are used at the same time, the video processing device 56 may be configured to select a view from one of the trocars 200, 300, 400 that is not actively being moved during teleoperation. As noted above, movement of the trocars 200, 300, 400 may be determined using a motion sensor or motion detection through a video feed provided by one of the trocars 200, 300, 400.

While the disclosure of using the trocar 200 was described with respect to the surgical robotic system 100, the trocar 200 along with the video processing device 56 and a display (e.g., display 23, 32, 34) may be used in alone or in combination with any other surgical system.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system, comprising:
a surgical robotic arm having an elongated rail configured to movably support a surgical instrument; and
a first trocar including:
a head configured for attachment to the elongated rail;
a cannula extending distally from the head and configured to receive the surgical instrument; and
a plurality of cameras disposed about a distal end portion of the cannula and directed radially outward.

2. The surgical robotic system according to claim 1, further comprising:
a video processing device in communication with the plurality of cameras of the first trocar, wherein the video processing device is configured to stitch together images taken by each of the plurality of cameras of the first trocar to form a single image; and
a display in communication with the video processing device and configured to display the single image.

3. The surgical robotic system according to claim 1, wherein the plurality of cameras is mounted to the distal end portion of the cannula in an annular array.

4. The surgical robotic system according to claim 1, wherein the first trocar includes a lens enclosing the plurality of cameras.

5. The surgical robotic system according to claim 1, wherein the distal end portion of the cannula defines a distal port, the plurality of cameras being disposed adjacent the distal port.

6. The surgical robotic system according to claim 1, wherein the first trocar includes at least one light disposed adjacent the plurality of cameras.

7. The surgical robotic system according to claim 1, further comprising a second trocar including:
a cannula defining a channel therethrough; and
a plurality of cameras disposed about a distal end portion of the cannula of the second trocar and directed radially outward.

8. The surgical robotic system according to claim 7, further comprising a video processing device in communication with the plurality of cameras of the first trocar and the second trocar, wherein the video processing device is configured to stitch together images taken by each of the plurality of cameras of the first and second trocars to form a single image and display the single image on a display.

9. The surgical robotic system according to claim 8, wherein the video processing device is further configured to focus the plurality of cameras of the first trocar or the second trocar on the other of the first trocar or the second trocar during insertion of the surgical instrument into the other of the first trocar or the second trocar.

10. A trocar for insertion into a body cavity, the trocar comprising:
a head defining an opening configured for receipt of a surgical instrument;
a cannula extending distally from the head and defining a channel configured for passage of the surgical instrument; and
a plurality of cameras disposed about a distal end portion of the cannula and directed radially outward.

11. The trocar according to claim 10, wherein the plurality of cameras is mounted to the distal end portion of the cannula in an annular array.

12. The trocar according to claim 11, wherein the cannula includes a lens enclosing the plurality of cameras.

13. The trocar according to claim 10, wherein the distal end portion of the cannula defines a distal port, the plurality of cameras mounted to the distal end portion of the cannula adjacent the distal port.

14. The trocar according to claim 10, wherein the trocar includes at least one light disposed adjacent the plurality of cameras.

15. The trocar according to claim 10, wherein the cannula has a proximal end portion attached to the head, the distal end portion having a distal tip configured for penetrating tissue.

16. A method of imaging an internal body cavity during a surgical procedure, the method comprising:
stitching images captured by a plurality of cameras disposed about a distal end portion of a first trocar to form a single image of a body cavity; and
displaying the single image of the body cavity on a display.

17. The method according to claim 16, further comprising at least one of:
activating the plurality of cameras of the first trocar as a second trocar is inserted into the body cavity; or
directing the plurality of cameras of the first trocar at the second trocar as the second trocar is inserted into the body cavity.

18. The method according to claim 17, further comprising detecting movement of a surgical instrument into the body cavity, whereupon the plurality of cameras of the first trocar and a plurality of cameras of the second trocar are oriented toward the surgical instrument.

19. The method according to claim 17, further comprising:
stitching together images taken by the plurality of cameras of the first trocar and a plurality of cameras of the second trocar to form a 3D image of the body cavity; and
display the 3D image on the display.

20. The method according to claim 16, further comprising illuminating the body cavity with a plurality of LEDs mounted to the distal end portion of the first trocar.
